(19) United States
(12) Patent Application Publication
(10) Pub. No.: US 2020/0318144 A1
(43) Pub. Date: Oct. 8, 2020

(54) METHOD AND DEVICE FOR STIMULATING BIOLOGICAL FERMENTATION

(71) Applicant: National Tsing Hua University, Hsinchu (TW)

(72) Inventors: JWO-HUEI JOU, Hsinchu City (TW); CHENG-CHIEH LO, Changhua County (TW); MING-RUEI JIANG, Tainan City (TW); YU-CHEN LAI, Hsinchu County (TW); CHUN-HO CHENG, Hsinchu City (TW); TING-WEN CHENG, Hsinchu City (TW)

(21) Appl. No.: 16/394,193

(22) Filed: Apr. 25, 2019

(30) Foreign Application Priority Data
Apr. 2, 2019 (TW) 108111686

(51) Int. Cl. C12P 7/06; C12M 1/00

(52) U.S. Cl. CPC C12P 7/06; C12M 41/06; C12M 31/02

(57) ABSTRACT

Disclosures of the present invention describe a method and device for stimulating biological fermentation. In the present invention, a light source is particularly used for stimulating a biological fermentation by supplying an illumination light with a color temperature in a range between 1600K and 4300K. Moreover, a variety of experimental data have proved that, the illumination light is indeed helpful in stimulating the biological fermentation occurring in an object under fermentation so as to enhance a rate of ethanol fermentation. It is worth further explaining that, the method and the device for stimulating biological fermentation can be applied in any one type of fermentation apparatus.

### BACKGROUND OF THE INVENTION

### 1. Field of the Invention

The present invention relates to the field of fermentation technologies, and more particularly to a method and device for stimulating biological fermentation.

### 2. Description of the Prior Art

Fermentation is a metabolic process that produces chemical changes in organic substrates through the action of enzymes. Therefore, the science of fermentation is known as zymology. In biochemistry, it is narrowly defined as the extraction of energy from carbohydrates in the absence of oxygen. In microorganisms, fermentation is the primary means of producing adenosine triphosphate (ATP) by the degradation of organic nutrients anaerobically. In the context of food production, however, it may more broadly refer to any process in which the activity of microorganisms brings about a desirable change to a foodstuff or beverage. Humans have used fermentation to produce foodstuffs and beverages since the Neolithic age. For example, fermentation is used for preservation in a process that produces lactic acid found in such sour foods as pickled cucumbers, kimchi, and yogurt, as well as for producing alcoholic beverages such as wine and beer.

Alternative energies have become increasingly important due to the foreseeable shortage of petrochemical energy. Amongst, biomass energies, including fuelwood, dung, crop residues, ethanol, and biogas, have been found can be the future major sources of fuel in the world. In which bio-alcohol (i.e., ethanol) is one good option at least as the fuel for automobiles. The conversion of raw materials containing sugars, starches or fibers to ethanol by yeast fermentation is so far one highly effective bio-alcohol production technology. Ethanol fermentation is carried out with yeast in an 8- to 12-h batch process, which produces 8 to 10% ethanol by volume.

Fermentation models could affect the production and conversion rate of bioethanol. Fermentation models of batch and fed-batch are mainly used for bioethanol production. For the traditional batch fermentation of sugar cane molasses with Saccharomyces cerevisiae, the produced bioethanol has a concentration of 80-100 g/L and a conversion rate of 85%˜90%, respectively. However, it is a pity that an entire processing time of the batch fermentation is very long, causing that the batch fermentation merely exhibits a bioethanol production rate in a range between 1 g/L·h and 3 g/L·h. On the other hand, despite the bioethanol production rate of the fed-batch fermentation is higher than that of the batch fermentation by 10-14%, the bioethanol produced by using the fed-batch fermentation only has the concentration of 53.7-92 g/L.

From above descriptions, it is clear how to effectively enhance the bioethanol production rate of the conventional fermentation technologies has become the most important issue. In view of that, inventors of the present application have made great efforts to make inventive research and eventually provided a method and device for stimulating biological fermentation.

### SUMMARY OF THE INVENTION

Conventional fermentation technology has known that can be adopted for production fermentation liquid containing ethanol with specific concentration by using yeast to apply a fermentation process to a specific raw material containing sugars, starches or fibers for 8-12 hours. However, an inadequate alcohol production per unit time (i.e., production rate) becomes a principal drawback of the conventional fermentation technology. Accordingly, the primary objective of the present invention is to disclose a method and device for stimulating biological fermentation. In the present invention, a light source is particularly used for stimulating a biological fermentation by supplying an illumination light with a color temperature in a range between 1600K and 4300K. A variety of experimental data have proved that, the illumination light is helpful in stimulating the biological fermentation occurring in an object under fermentation so as to enhance a rate of ethanol fermentation. Moreover, the present invention also discloses a device for stimulating biological fermentation, which can be applied in any one type of fermentation apparatus.

In order to achieve the primary objective of the present invention, the inventor of the present invention provides an embodiment for the method for stimulating biological fermentation, comprising following steps:
providing a light source;
controlling the light source to emit an illumination light with a color temperature in a range between 1600K and 4300K; and
guiding the illumination light into a fermentation apparatus accommodating an object under fermentation, so as to make the object under fermentation receive the illumination light, thereby enhancing rate of ethanol fermentation by at least 25%.

Moreover, the inventor of the present invention also provides an embodiment for the device for stimulating biological fermentation, which comprises a light source, wherein the light source is configured for emitting an illumination light with a color temperature in a range between 1600K and 4300K, and a rate of ethanol fermentation of an object under fermentation being enhanced by at least 25% after the is exposed under the illumination light.

In one embodiment of the device, the light source comprises at least one lighting device selected from the group consisting of incandescent lamp, fluorescent light, light-emitting diode (LED), quantum dot light-emitting diode (QD-LED), and organic light emitting diode (OLED).

In one embodiment of the device, the illumination light is a multi-band light and has a luminance lower than 750 lx.

In another one embodiment of the device, there is a driver module further provided, and the light source comprises a plurality of lighting devices that are configured for being controlled by the driver module, such that a plurality of lights are respectively emitted from the plurality of lighting devices so as to form the illumination light.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a first stereo diagram of a fermentation apparatus with a device for stimulating biological fermentation according to the present invention;

FIG. 2 shows a stereo diagram of a fermenting liquid culturing tank;

FIG. 3 shows a stereo diagram of a temperature-controlled fermentation box;

FIG. 4 shows a second stereo diagram of the fermentation apparatus with a device for stimulating biological fermentation according to the present invention;

FIG. 5 shows a second stereo diagram of an experiment framework;

FIG. 6 shows an exploded diagram of the experiment framework;

FIG. 7 shows a light spectrum graph of a first light;

FIG. 8 shows a curve graph of fermentation time versus CO₂ volume;

FIG. 9 shows a light spectrum graph of the first light, a second light, a third, and a fourth light; and

FIG. 10 shows a curve graph of fermentation time versus CO₂ volume.

### DETAILED DESCRIPTION OF THE PREFERRED EMBODIMENTS

To more clearly describe a method and device for stimulating biological fermentation disclosed by the present invention, embodiments of the present invention will be described in detail with reference to the attached drawings hereinafter.

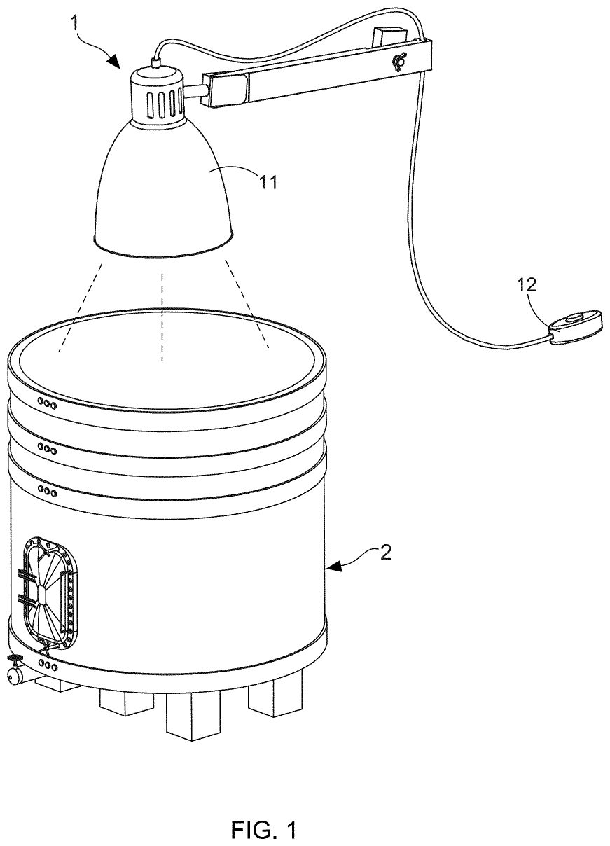

With reference to FIG. 1, there is shown a first stereo diagram of a fermentation apparatus with a device for stimulating biological fermentation according to the present invention. As FIG. 1 shows, the fermentation apparatus 2 is a wine brewing barrel, and the biological fermentation stimulating device 1 of the present invention comprises a light source 11 and a driver module 12. According to the particular design of the present invention, the light source 11 is configured for being controlled by the driver module 12, so as to emit an illumination light with color temperature in a range between 1600K and 4300K. After being measured, the illumination light is known to be a multi-band light, and has a luminance lower than 750 lx.

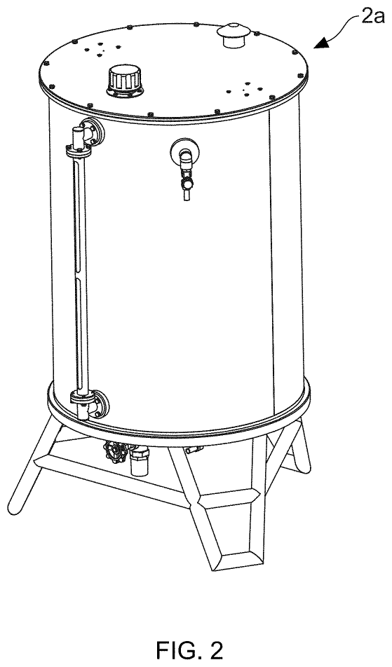

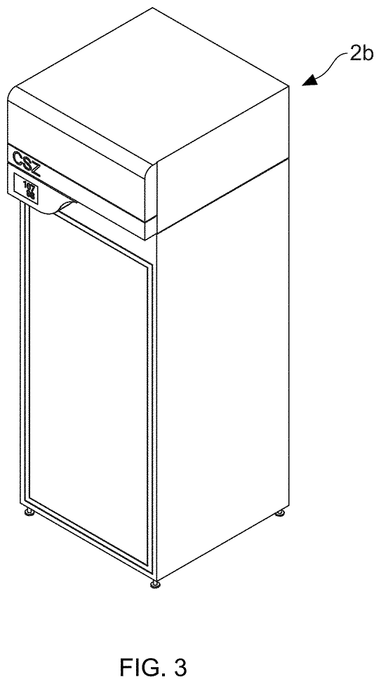

Herein, it needs to particularly emphasize that, this biological fermentation stimulating device 1 can be used in combination with any one type of fermentation apparatus 2. Briefly speaking, the said fermentation apparatus 2 is not limited to be the wine brewing barrel of FIG. 1. For example, this biological fermentation stimulating device 1 can also be used in combination with either a fermenting liquid culturing tank 2a shown in FIG. 2 or a temperature-controlled fermentation box 2b shown in FIG. 3. As described in detail below, the fermenting liquid culturing tank 2a of FIG. 2 is commonly adopted for completing mass production of a specific fermenting liquid. Therefore, after putting a specific yeast and suitable raw materials containing sugars, starches or fibers into the culturing tank 2a for carrying out a fermentation process, the biological fermentation stimulating device 1 of the present invention can be simultaneously adopted for supplying the illumination light with the color temperature of 1600-4300K to those object under fermentation, thereby enhancing a bioethanol production rate of the fermentation process.

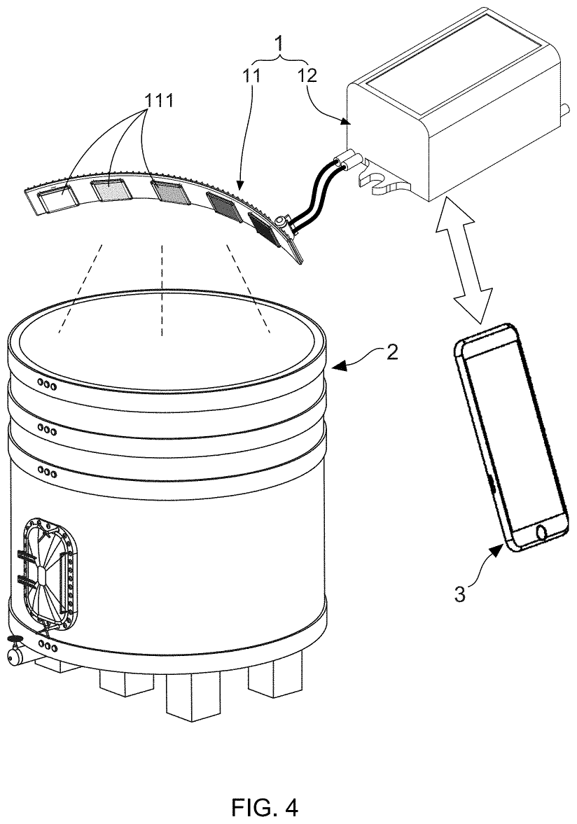

In one embodiment, as shown in FIG. 1, the light source comprises at least one lighting device (or element), and which can be an incandescent lamp, a fluorescent light, a light-emitting diode (LED), a quantum dot light-emitting diode (QD-LED), and an organic light emitting diode (OLED). However, inventors of the present invention have found that, rate of ethanol fermentation of the object under fermentation varies with the irradiation of different illumination lights having different color temperature values (1600-4300K). Accordingly, as shown in FIG. 4, the present invention further discloses that the light source 11 of the biological fermentation stimulating device 1 can also be configured to comprises a plurality of lighting elements 111. By such arrangement, a plurality of lights are respectively emitted from the plurality of lighting elements 111 that are controlled by the driver module 12, and then the plurality of lights together form the illumination light.

As described in detail below, the light source 11 are configured to have five lighting elements 111, which are able to respectively emit a first light with first color temperature, a second light with second color temperature, a third light with third color temperature, a fourth light with fourth color temperature, and a fifth light with fifth color temperature. Moreover, each of the five lights is a multi-band light and has a luminance lower than 750 lx. On the other hand, any two of the five color temperature values are different from each other, but the color temperature values are all fall in a range between 1600K and 4300K. Therefore, it is easily understood that, an electronic device 3 can be used for being as a human-machine interface (HMI) that allows a user to control one or more lighting elements to emit their light, in order to adaptively modulating the bioethanol production rate and an entire processing time of the fermentation process that is applied to the object under fermentation.

Experiments

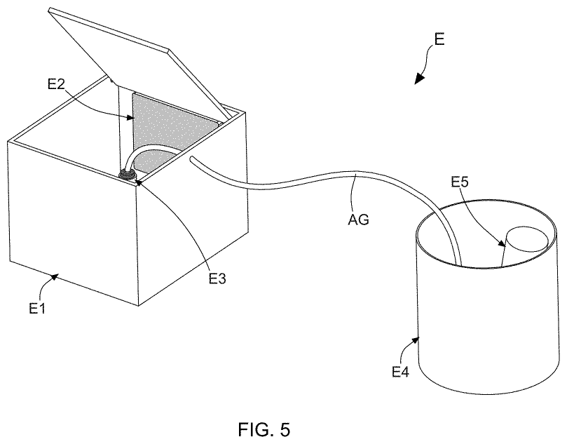

For proving that the illumination light with the color temperature in a range between 1600K and 4300K can indeed be helpful for enhancing the rate of ethanol fermentation of an object under fermentation, inventors of the present invention have completed several experiments. FIG. 5 illustrates a second stereo diagram of an experiment framework, and FIG. 6 shows an exploded view of the experiment framework. As FIG. 5 and FIG. 6 show, the experiment framework E consists of a dark box E1, a lighting unit E2, an erlenmeyer flask E3, a beaker E4, and a graduated cylinder E5, wherein the lighting unit E2 and the erlenmeyer flask E3 are disposed in the dark box E1. Moreover, there is a malt extract inoculated with yeast accommodated in the erlenmeyer flask E3, wherein the malt extract is obtained by mixing power of malt extract with water. On the other hand, the graduated cylinder E5 is putted into the beaker E4 filled with water by an opening end thereof, and two terminals of an air transmitting tube AG are connected to the opening end of the graduated cylinder E5 and putted into the erlenmeyer flask E3, respectively.

It should know that, fermentation is an anaerobic respiration of yeast that happens in an environment (i.e., the erlenmeyer flask E3) without oxygen. The reaction of fermentation can be shown as following:

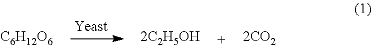

From the foregoing reaction equation, it is understood that two alcohol molecules and two carbon dioxides molecules can be generated after the fermentation of one glucose molecule via yeast. Hence, the collected carbon dioxide (CO₂) can be used to estimate the produced alcohol and the rate of fermentation. Briefly speaking, the beaker E4, the air transmitting tube AG and the graduated cylinder E5 are arranged for collecting carbon dioxide (CO₂) produced in the erlenmeyer flask E3 through a drainage gas collection method.

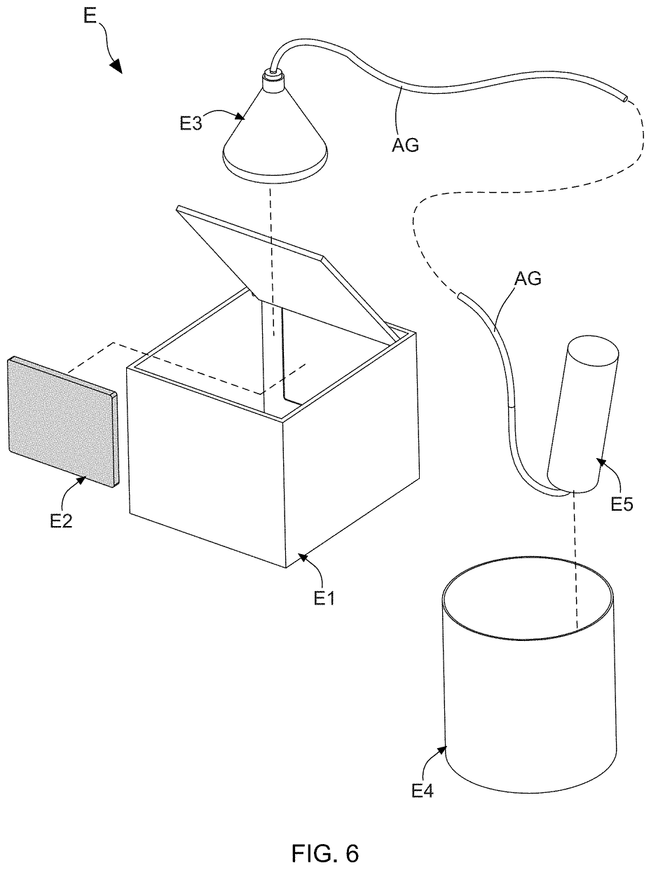

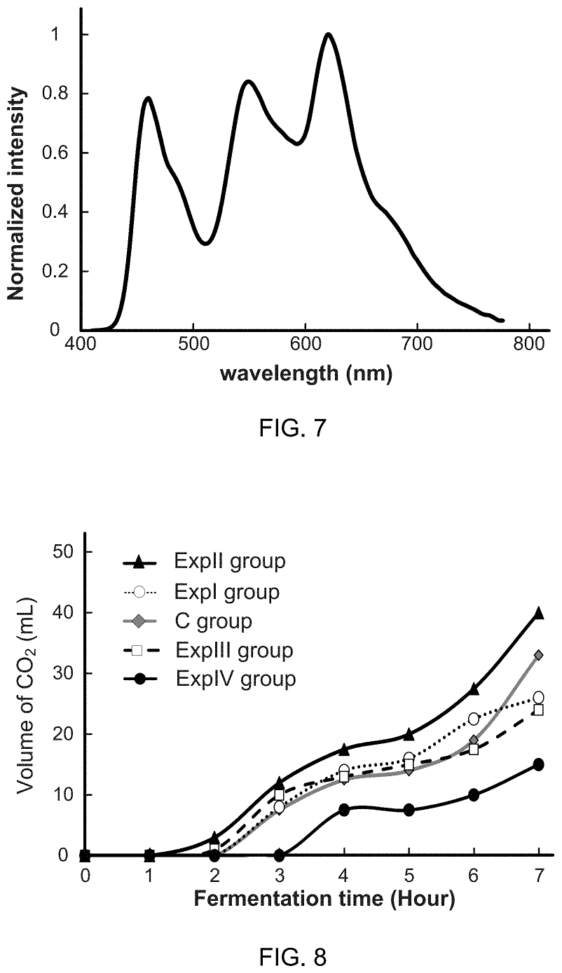

In a first experiment, the lighting unit E2 is configured for emit a first light with color temperature of 4000K, and FIG. 7 shows a light spectrum graph of the first light. There are one control (C) group and four experimental (Exp) groups properly designed for completing the first experiment. Related information about the C group and the four Exp groups are summarized in following Table (1).

TABLE (1)

| Groups | Test sample | Descriptions |
|---|---|---|
| C | Malt extract | After putting the test sample into |
| | inoculated with yeast | the erlenmeyer flask E3, and the |
| | | erlenmeyer flask E3 is subsequently |
| | | putted into the dark box. |
| | | Consequently, the test sample is |
| | | applied with a dark culture process |
| | | for 6-7 hours. |
| ExpI | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a first light |
| | | with color temperature of 4000K to |
| | | the test sample, the test sample is |
| | | applied with a light culture process |
| | | for 6-7 hours. In which the first light |
| | | has a luminance of 250 lx. |
| ExpII | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a first light |
| | | with color temperature of 4000K to |
| | | the test sample, the test sample is |
| | | applied with a light culture process |
| | | for 6-7 hours. In which the first light |
| | | has a luminance of 500 lx. |
| ExpIII | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a first light |
| | | with color temperature of 4000K to |
| | | the test sample, the test sample is |
| | | applied with a light culture process |
| | | for 6-7 hours. In which the first light |
| | | has a luminance of 750 lx. |
| ExpIV | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a first light |
| | | with color temperature of 4000K to |
| | | the test sample, the test sample is |
| | | applied with a light culture process |
| | | for 6-7 hours. In which the first light |
| | | has a luminance of 1000 lx. |

FIG. 8 shows a curve graph of fermentation time versus CO₂ volume. From the experimental data of FIG. 8, it is found that the volume of CO₂ collected from the erlenmeyer flask E3 in ExpII group is higher than the volume of CO₂ collected from the erlenmeyer flask E3 in C group. Moreover, FIG. 8 also reveals that the volume of CO₂ collected from either the ExpIII group or the ExpIV group is lower than the volume of CO₂ collected from the C group. Therefore, related data obtained from the first experiment have proved that, a specific illumination light having a color temperature of 4300K and a luminance lower than 750 lx is helpful in stimulating the bioethanol production rate and/or the rate of ethanol fermentation in an object under fermentation.

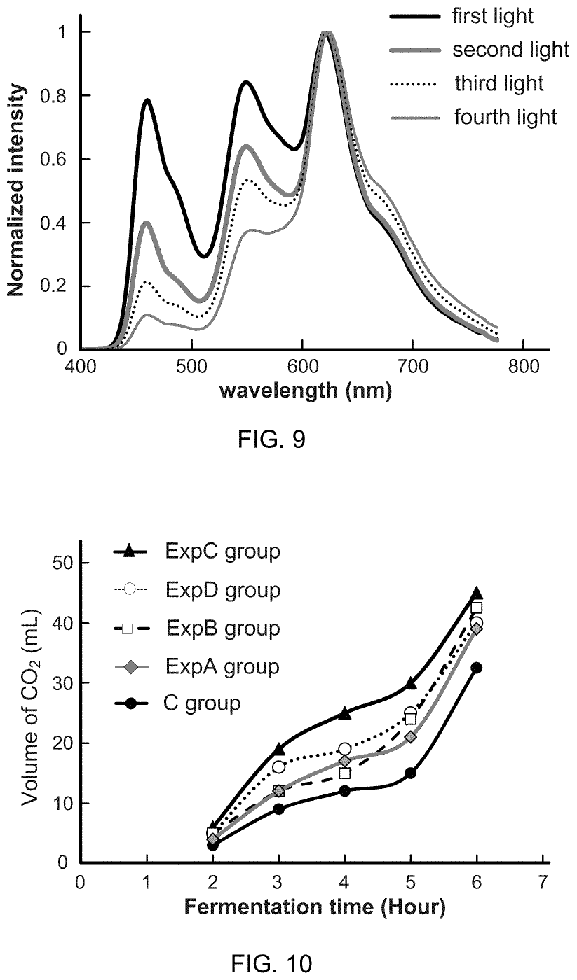

In a second experiment, the lighting unit E2 is configured to emit a first light with first color temperature of 4000K, a second light with second color temperature of 3000K, a third light with third color temperature of 2300K, and a fourth light with fourth color temperature of 1900K. FIG. 9 shows a light spectrum graph of the first light, a second light, a third, and a fourth light, and also depicts that each of the four lights is a multi-band light. There are one control (C) group and four experimental (Exp) groups properly designed for completing the second experiment. Related information about the C group and the four Exp groups are summarized in following Table (2).

TABLE (2)

| Groups | Test sample | Descriptions |
|---|---|---|
| C | Malt extract | After putting the test sample into |
| | inoculated with yeast | the erlenmeyer flask E3, and the |
| | | erlenmeyer flask E3 is subsequently |
| | | putted into the dark box. |
| | | Consequently, the test sample is |
| | | applied with a dark culture process |
| | | for 6-7 hours. |
| ExpA | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a first light |
| | | with color temperature of 4000K to |
| | | the test sample, the test sample is |
| | | applied with a light culture process |
| | | for 6-7 hours. In which the first light |
| | | has a luminance of 500 lx. |
| ExpB | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a second |
| | | light with color temperature of |
| | | 3000K to the test sample, the test |
| | | sample is applied with a light culture |
| | | process for 6-7 hours. In which the |
| | | second light has a luminance of 500 lx. |
| ExpC | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a third light |
| | | with color temperature of 2300K to |
| | | the test sample, the test sample is |
| | | applied with a light culture process |
| | | for 6-7 hours. In which the third light |
| | | has a luminance of 500 lx. |
| ExpD | Malt extract | After putting the test sample into |
| | inoculated with yeast | the erlenmeyer flask E3, and the |
| | | erlenmeyer flask E3 is subsequently |
| | | putted into the dark box. |
| | | Consequently, by configuring the |
| | | lighting unit E2 to supply a fourth |
| | | light with color temperature of |
| | | 1900K to the test sample, the test |
| | | sample is applied with a light culture |
| | | process for 6-7 hours. In which the |
| | | fourth light has a luminance of 500 lx. |

FIG. 10 shows a curve graph of fermentation time versus CO₂ volume. From the experimental data of FIG. 10, it is found that the volume of CO₂ collected from any one of the four Exp groups is higher than the volume of CO₂ collected from the C group. Particularly, the volume of CO₂ collected from the ExpC group is higher than the volume of CO₂ collected from the C group by 38%, and the volume of CO₂ collected from the ExpA group is higher than the volume of CO₂ collected from the C group by 28%. Therefore, related data obtained from the first experiment have proved that, a specific illumination light having a color temperature and a luminance lower than 750 lx is helpful in enhancing rate of ethanol fermentation in an object under fermentation by at least 25%. In which the color temperature is in a range between 1600K and 4300K.

Therefore, through above descriptions, all embodiments and their constituting elements of the device for stimulating biological fermentation proposed by the present invention have been introduced completely and clearly; in summary, the present invention includes the advantages of:

(1) Conventional fermentation technology has known that can be adopted for production fermentation liquid containing ethanol with specific concentration by using yeast to apply a fermentation process to a specific raw material containing sugars, starches or fibers for 8-12 hours. However, an inadequate alcohol production per unit time (i.e., production rate) becomes a principal drawback of the conventional fermentation technology. Accordingly, the primary objective of the present invention is to disclose a method and device for stimulating biological fermentation. In the present invention, a light source 11 is particularly used for stimulating a biological fermentation by supplying an illumination light with a color temperature in a range between 1600K and 4300K. A variety of experimental data have proved that, the illumination light is helpful in stimulating the biological fermentation occurring in an object under fermentation so as to enhance a rate of ethanol fermentation. Moreover, the disclosed biological fermentation stimulating device 1 can be applied in any one type of fermentation apparatus.

The above description is made on embodiments of the present invention. However, the embodiments are not intended to limit scope of the present invention, and all equivalent implementations or alterations within the spirit of the present invention still fall within the scope of the present invention.

What is claimed is:

1. A method for stimulating biological fermentation, comprising following steps:
providing a light source;
controlling the light source to emit an illumination light with a color temperature in a range between 1600K and 4300K; and
guiding the illumination light into a fermentation apparatus accommodating an object under fermentation, so as to make the object under fermentation receive the illumination light, thereby enhancing rate of ethanol fermentation by at least 25%.

2. The method of claim 1, wherein the light source comprises at least one lighting device selected from the group consisting of incandescent lamp, fluorescent light, light-emitting diode (LED), quantum dot light-emitting diode (QD-LED), and organic light emitting diode (OLED).

3. The method of claim 1, wherein the illumination light has a luminance lower than 750 lx.

4. The method of claim 1, wherein the illumination light is a multi-band light.

5. A device for stimulating biological fermentation, comprising a light source, wherein the light source is configured for emitting an illumination light with a color temperature in a range between 1600K and 4300K, and a rate of ethanol fermentation of an object under fermentation being enhanced by at least 25% after the is exposed under the illumination light.

6. The device of claim 5, wherein the light source comprises at least one lighting device selected from the group consisting of incandescent lamp, fluorescent light, light-emitting diode (LED), quantum dot light-emitting diode (QD-LED), and organic light emitting diode (OLED).

7. The device of claim 5, wherein the illumination light has a luminance lower than 750 lx.

8. The device of claim 5, wherein the illumination light is a multi-band light.

9. The device of claim 5, further comprising a driver module, and the light source comprises a plurality of lighting devices that are configured for being controlled by the driver module, such that a plurality of lights are respectively emitted from the plurality of lighting devices so as to form the illumination light.

10. The device of claim 9, wherein the driver module communicates with an electronic device selected from the group consisting of desk computer, laptop computer, industrial computer, server computer, smart phone, tablet PC, and smart watch.
